(19) United States
(12) Patent Application Publication
(10) Pub. No.: US 2026/0248447 A1
(43) Pub. Date: Aug. 27, 2026

(54) ARTIFICIAL INTELLIGENCE-BASED METHOD AND SYSTEM FOR COGNITIVE ASSESSMENT THROUGH SPEECH ANALYSIS

(71) Applicant: SENSORY SCIENCE INC., Seneca, SC (US)

(72) Inventor: Maurice Edward DURSCHLAG, Seneca, SC (US)

(21) Appl. No.: 19/548,760

(22) Filed: Feb. 24, 2026

Related U.S. Application Data
(60) Provisional application No. 63/834,129, filed on Feb. 24, 2025.
(60) Provisional application No. 63/808,344, filed on May 19, 2025.
(60) Provisional application No. 63/841,575, filed on Jul. 10, 2025.
(60) Provisional application No. 63/847,805, filed on Jul. 21, 2025.

(51) Int. Cl. A61B 5/00; G10L 15/02; G10L 15/18; G16H 50/20; G16H 50/30

(52) U.S. Cl. CPC A61B 5/4803; A61B 5/4088; A61B 5/7267; G10L 15/02; G10L 15/1807; G10L 15/1815; G16H 50/20; G16H 50/30

(57) ABSTRACT

A method and non-transitory computer-readable medium for generating cognitive assessment scores from patient speech using an artificial-intelligence (“AI”) agent is provided. The method and computer-readable medium include initiating a patient audio communication session, conducting one or more oral cognitive assessments including narrative recall, engaging in a context-dependent discussion phase, assessing responses, and generating scores from audiometric, lexical, prosodic, and semantic-coherence features using multi-modal machine learning architectures. Audio data is processed by a backend analysis server using automatic speech recognition to generate a time-aligned transcript, and the backend analysis server uses a feature-embedding model to extract prosodic, acoustic, lexical, and semantic-coherence features from the audio and corresponding transcript. The backend analysis server uses machine-learning classification to generate cognitive assessment scores from extracted features. Assessment scores, feature data, and session records may be transmitted to clinician-accessible locations for review. The method enables automated, scalable, and reproducible speech-based patient cognitive assessments on patient-accessible devices.

### RELATED APPLICATIONS

This application claims the benefit of priority to U.S. Provisional Application No. 63/834,129, filed Feb. 24, 2025, U.S. Provisional Application No. 63/808,344, filed May 19, 2025, U.S. Provisional Application No. 63/841,575, filed Jul. 10, 2025, and U.S. Provisional Application No. 63/847,805, filed Jul. 21, 2025, the contents of which are incorporated by reference herein in their entirety.

### BACKGROUND

Cognitive function assessment is an essential step in diagnosing, monitoring, and managing many neurological and psychiatric conditions. Speech-based features such as prosodic changes, lexical patterns, semantic coherence, and acoustic variability can reflect underlying neurological changes, which enable accurate measurement of patient impairments. Ongoing artificial intelligence (“AI”) advancements, including conversational AI and automated speech-analysis algorithms, create opportunities for early cognitive change detection through patient speech analysis.

### SUMMARY

The subject matter described herein includes a method and non-transitory computer-readable medium for automated cognitive assessment and generation of cognitive assessment scores from patient speech. In Example 1, a method for automated cognitive assessment and generation of cognitive assessment scores from patient speech includes a server that hosts an artificial intelligence (AI) conversational agent. Using the server, the method first initiates an audio communication session with a patient. Then, the method uses the AI conversational agent to deliver an oral narrative to the patient, which is selected from a library of clinically validated narratives. The method uses the AI conversational agent to deliver one or more oral cognitive assessment to the patient, including but not limited to categorical or letter verbal fluency, short-term memory word recall, text descriptions, and picture descriptions. The method further uses the AI conversational agent to deliver one or more immediate recall audio prompts to prompt the patient to orally recall information from the oral narrative. The method then uses the AI conversational agent to engage the patient in an oral topic discussion phase that incorporates targeted follow-up questions which are dynamically generated based on the patient's responses. After engaging the patient in the oral topic discussion phase, the method uses the AI conversational agent to deliver one or more delayed recall audio prompts to prompt the patient to orally recall information from the oral narrative. The method collects a collection of audio data retrieved during the audio communication session. The method can generate a session record from collected audio communication session data, the audio communication session data comprising device diagnostic information, patient identifying information, session metadata, and environmental information in a session record on a backend analysis server. The method utilizes a backend analysis server to generate a time-aligned transcript from the collection of audio data using an automatic speech recognition (ASR) model, wherein words in the transcript are associated with corresponding audio data timestamps of the collection of audio data. Then the method uses the backend analysis server to extract a plurality of speech-derived features from the collection of audio data and time-aligned transcript using a feature-embedding model, wherein the plurality of speech-derived features comprise but are not limited to prosodic features, acoustic characteristics, lexical features, and semantic-coherence metrics. The method next uses the backend analysis server to generate at least one cognitive assessment score from the plurality of speech-derived features using a machine-learning classifier trained on clinically validated datasets, wherein generating the at least one cognitive assessment score includes comparing the plurality of speech-derived features to diagnostic indicators. Finally, the method uses the backend analysis server to transmit the at least one cognitive assessment score, and session record if present, to a clinician-accessible device.

Example 2 includes the method of Example 1, wherein selection of the oral narrative is based on patient metadata including demographics or existing impairment indicators provided by an ordering physician.

Example 3 includes the method of Example 1, wherein the immediate and delayed recall audio prompts relate to semantic units comprising settings, characters, events, and thematic elements extracted from the oral narrative.

Example 4 includes the method of Example 1, wherein oral topic discussions conducted by the AI conversational agent include oral discussion topics generated based on patient responses and metadata, and in which follow-up queries are generated based on previous patient responses, and wherein oral discussion topics are generated from the group consisting essentially of the patient's hobbies, family matters, occupation, daily activities, wellbeing, and personal opinions.

Example 5 includes the method of Example 1, wherein the backend analysis server obtains patient session feedback prior to termination of the audio communication session.

Example 6 includes the method of Example 1, wherein a generated session record includes session data comprising a collection of audio data, cognitive assessment results, topic-phase data, device information, environmental data, session metadata, and patient identifiers.

Example 7 includes the method of Example 1, wherein a feature-embedding model is configured to generate feature vectors comprising articulation rates, speech segmentation, voice onset time, Mel-frequency Cepstral Coefficients, fundamental frequency, jitter, shimmer, contextual relevance metrics, harmonics-to-noise ratio, and formant.

Example 8 includes the method of Example 1, wherein a machine-learning classifier is trained to generate at least one cognitive assessment score for the measurement and prediction of disorders and conditions including but not limited to Mild Cognitive Impairment, Alzheimer's Disease, preclinical Alzheimer's Disease, age-related cognitive decline, Vascular Dementia, Dementia with Lewy Bodies, Frontotemporal Dementia, Parkinson's Disease with or without Dementia, Depression, Stroke, Huntington's Disease, Fatal Familial Insomnia, Traumatic Encephalopathy, and Normal Pressure Hydrocephalus.

Example 9 includes the method of Example 1, wherein the generated at least one cognitive assessment score includes at least one of an impairment probability score, risk of impairment probability score, or an impairment etiology (e.g., Alzheimer's disease, vascular contributions, depression) probability score.

Example 10 includes the method of Example 1, wherein the generated at least one cognitive assessment score includes at least one of a lexical complexity score, fluency control score, lexical diversity score, grammatical sophistication score, executive control score, descriptiveness score, content accuracy score, coherence score, and scores for cognitive domains including memory, executive functioning, language use, visuospatial processing, and attention.

Example 11 includes the method of Example 1, wherein the generated at least one cognitive assessment score includes transparent explanatory data and human-in-the-loop control elements compliant with the EU AI Act, FDA PCCP, YY/T 1833, or equivalent Artificial Intelligence system design and security regulations and standards.

Example 12 includes the method of Example 1, wherein transmitted data includes standardized clinical data fields compliant for integration with an electronic health records system.

Example 13 includes the method of Example 1, wherein data erasure complies with SP 800-88 or equivalent media sanitization standards.

Example 14 includes the method of Example 1, wherein integrations with electronic health records systems comply with HL7 Fast Healthcare Interoperability Resources standards, HIPAA, YY/T 1843, GDPR, or equivalent medical data privacy and security regulations and standards.

Example 15 includes the method of Example 1, wherein collected and transmitted data is encrypted using cryptographic modules compliant with FIPS 140-3 Level 2 or equivalent data security standards.

Example 16 includes the method of Example 1, wherein a feedback-based training procedure is executed using clinician-generated feedback and diagnosis data to update parameters of at least one model used by the method.

In Example 17, the non-transitory computer-readable medium for generating cognitive assessment scores from patient speech includes instructions executable by one or more processing devices. The instructions' execution by the one or more processing device initiates an artificial intelligence (AI) based cognitive assessment system. The implemented cognitive assessment system uses a server hosting an AI conversational agent to initiate an audio communication session with a patient. The system uses the AI conversational agent to deliver an oral narrative to the patient, which is selected from a library of clinically validated narratives. The system uses the AI conversational agent to deliver one or more oral cognitive assessments to the patient, including but not limited to categorical or letter verbal fluency, short-term memory word recall, text descriptions, and picture descriptions. The system further uses the AI conversational agent to deliver one or more immediate recall audio prompts to prompt the patient to orally recall information from the oral narrative. The system then uses the AI conversational agent to engage the patient in an oral topic discussion phase that incorporates targeted follow-up questions which are dynamically generated based on the patient's responses. After engaging the patient in a topic discussion phase, the system uses the AI conversational agent to deliver one or more delayed recall audio prompts to prompt the patient to orally recall information from the oral narrative. The system collects a collection of audio data retrieved during the audio communication session. The system can generate a session record from collected audio communication session data, the audio communication session data comprising device diagnostic information, patient identifying information, session metadata, and environmental information in a session record on a backend analysis server. The system utilizes the backend analysis server to generate a time-aligned transcript from the collection of audio data using an automatic speech recognition (ASR) model, wherein words in the transcript are associated with corresponding audio data timestamps of the collection of audio data. Then the system uses the backend analysis server to extract a plurality of speech-derived features from the collection of audio data and time-aligned transcript using a feature-embedding model, wherein the plurality of speech-derived features comprise but are not limited to prosodic features, acoustic characteristics, lexical features, and semantic-coherence metrics. The system next uses the backend analysis server to generate at least one cognitive assessment score from the plurality of speech-derived features using a machine-learning classifier trained on clinically validated datasets, wherein generating the at least one assessment score includes comparing a plurality of speech-derived features to diagnostic indicators. Finally, the system uses the backend analysis server to transmit the cognitive assessment score, and session record if present, to a clinician-accessible device.

Example 18 includes the non-transitory computer-readable medium of Example 17, wherein selection of the oral narrative is based on patient metadata including demographics or existing impairment indicators provided by an ordering physician.

Example 19 includes the non-transitory computer-readable medium of Example 17, wherein oral topic discussions conducted by the AI conversational agent include oral discussion topics generated based on patient responses and metadata, and in which follow-up queries are generated based on previous patient responses, and wherein oral discussion topics are generated from the group consisting essentially of the patient's hobbies, family matters, occupation, daily activities, wellbeing, and personal opinions.

Example 20 includes the non-transitory computer-readable medium of Example 17, wherein a feature-embedding model is configured to generate feature vectors comprising articulation rates, speech segmentation, voice onset time, Mel-frequency Cepstral Coefficients, fundamental frequency, jitter, shimmer, contextual relevance metrics, harmonics-to-noise ratio, and formant.

Example 21 includes the non-transitory computer-readable medium of Example 17, wherein a machine-learning classifier is trained to generate at least one cognitive assessment score for the measurement and prediction of disorders and conditions including but not limited to Mild Cognitive Impairment, Alzheimer's Disease, preclinical Alzheimer's Disease, age-related cognitive decline, Vascular Dementia, Dementia with Lewy Bodies, Frontotemporal Dementia, Parkinson's Disease with or without Dementia, Depression, Stroke, Huntington's Disease, Fatal Familial Insomnia, Traumatic Encephalopathy, and Normal Pressure Hydrocephalus.

Example 22 includes the non-transitory computer-readable medium of Example 17, wherein the generated at least one cognitive assessment score includes at least one of an impairment probability score, risk of impairment probability score, or an impairment etiology (Alzheimer's disease, vascular contributions, depression) probability score.

Example 23 includes the non-transitory computer-readable medium of Example 17, wherein the generated at least one cognitive assessment score includes at least one of a lexical complexity score, fluency control score, lexical diversity score, grammatical sophistication score, executive control score, descriptiveness score, content accuracy score, coherence score, and scores for cognitive domains including memory, executive functioning, language use, visuospatial processing, and attention.

Example 24 includes the non-transitory computer-readable medium of Example 17, wherein the generated at least one cognitive assessment score includes transparent explanatory data and human-in-the-loop control elements compliant with the EU AI Act, FDA PCCP, YY/T 1833, or equivalent Artificial Intelligence system design and security regulations and standards

Example 25 includes the non-transitory computer-readable medium of Example 17, wherein transmitted data includes standardized clinical data fields compliant for integration with an electronic health records system.

Example 26 includes the non-transitory computer-readable medium of Example 17, wherein data erasure complies with SP 800-88 or equivalent media sanitization standards.

Example 27 includes the non-transitory computer-readable medium of Example 17, wherein integrations with electronic health records systems comply with HL7 Fast Healthcare Interoperability Resources standards, HIPAA, YY/T 1843, GDPR, or equivalent medical data privacy and security regulations and standards.

Example 28 includes the non-transitory computer-readable medium of Example 17, wherein collected and transmitted data is encrypted using cryptographic modules compliant with FIPS 140-3 Level 2 or equivalent data security standards.

Example 29 includes the non-transitory computer-readable medium of Example 17, wherein a session record is generated from audio communication session data comprising collected audio data from the one or more cognitive assessment, device diagnostic information, patient identifying information, session metadata, environmental information, cognitive assessment results, topic-phase data, device information, environmental data, session metadata, and patient identifiers.

### BRIEF DESCRIPTION OF THE DRAWINGS

Understanding that the drawings depict only exemplary embodiments and are not therefore to be considered limiting in scope, the exemplary embodiments will be described with additional specificity and detail in the accompanying drawings, in which:

FIG. 1 is a block diagram of an example system including an AI conversational agent, a patient using a patient-operated communications device with associated audio-capture components, and related backend system servers.

FIG. 2 is a flow diagram of an example audio-based conversational assessment session illustrating narrative selection and delivery, assessment prompting, and context-dependent topic discussions.

FIG. 3 is a flow diagram of an example process for generating conversational discussion topics, including narrative selection, dynamic topic generation, and dynamic follow-up topic generation.

FIG. 4 is a flow diagram of an example method for generating cognitive assessment scores using acquired audio data, ASR-generated transcripts, speech-derived feature extraction, and machine-learning-based classifiers.

FIG. 5 is a block diagram of an example patient-accessible communications device configured to execute or interface with the AI conversational agent, such as a telephone, mobile phone, tablet, computer, wearable device, or virtual home assistant.

### DESCRIPTION

Traditional cognitive assessments are frequently conducted through in-person evaluations and structured interviews, which typically require trained clinicians, rely on subjective interpretation, and may be difficult to perform regularly or at-scale. Further, many individuals lack convenient access to clinical facilities, which may delay recognition of cognitive decline. Existing clinical workflows rarely incorporate automated extraction of speech-derived clinical diagnostic features, and available tools are typically not scalable, require manual audio transcription, and/or cannot be deployed in convenient and accessible environments such as the patient's home. Existing approaches often fail to capture subtle, early-stage symptoms, as mild cognitive impairment and early signs of neurodegenerative disorders frequently appear gradually in speech patterns and behaviors, and such early indicators may be missed during infrequent clinical encounters. Current systems generally do not offer continuous, real-world cognitive function monitoring, and many patients thereby lose early intervention opportunities. Further, existing systems struggle to extract clinically meaningful speech features in situations involving varied environmental noise conditions, varied conversation dynamics, and spontaneous utterance patterns.

There is therefore a need for improved, patient-accessible systems that collect and analyze patient speech using objective, automated, and reproducible methods. Such systems should enable non-invasive cognitive assessment via mobile devices, tablets, computers, virtual home assistants, and other dedicated collection hardware incorporating voice-recognition, noise isolation, and/or source-separation capabilities. The improved systems should further support population data comparisons, integrate with clinical workflows, and facilitate early detection of cognitive impairment and measurement of current cognitive functioning through continuous or periodic monitoring without the need for specialized personnel intervention. Improved systems should additionally provide affirmative population screening capabilities, longitudinal monitoring of cognitive assessment data, and means to implement real-time clinician alerts for early intervention. The improved systems should enable clinician access to historic and current assessment data via standardized healthcare data systems and via remote or resource-constrained systems.

### DRAWINGS

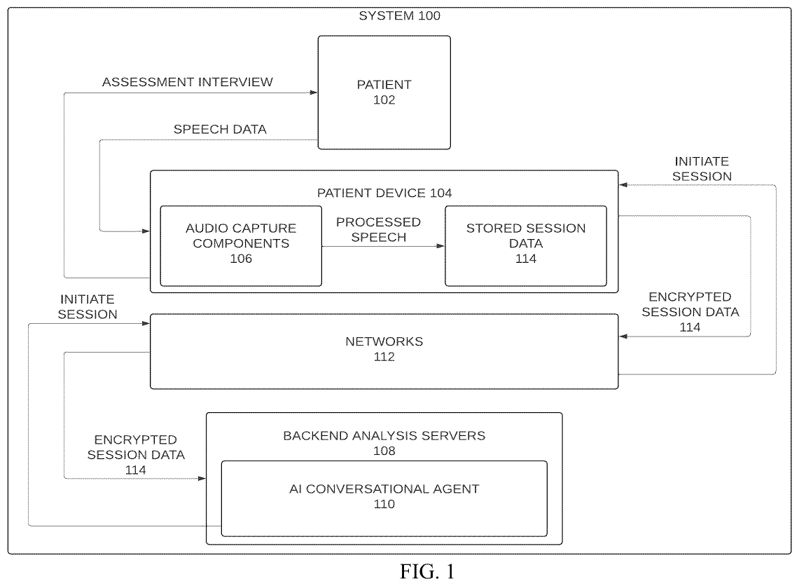

FIG. 1 is a block diagram of an example system 100 for conducting AI-driven cognitive assessment communication sessions. The system 100 comprises a patient 102 operating a patient-controlled device 104, which includes audio-capture components 106, and a plurality of backend analysis servers 108 that are communicatively coupled with the patient device 104. The system further comprises an AI conversational agent 110 executed by software and communicatively coupled to the patient device 104 and the backend analysis servers 108. The AI conversational agent 110, the patient device 104, and the backend analysis servers 108 can be communicatively coupled together over one or more networks 112, including, but not limited to, a landline-based telephone network, a cellular communications network, a local area network, intranet, and/or the internet. Communication between the system's 100 components can use any appropriate protocol including Signaling System No. 7(SS7), Q.931, Internet Protocol (IP), H.323, Voice Over IP (VOIP), Hypertext Transfer Protocol (HTTP), Hypertext Transfer Protocol Secure (HTTPS), and other telephonic- and internet-based protocols. Session data 114 may be locally or remotely stored depending on implementation. All patient data will be encrypted and maintained in an access-controlled, audit-enabled secure directory to ensure compliance with HIPAA, FDA ERES, SP 800-111, SP 800-122, YY/T 1843, GDPR, or equivalent healthcare data privacy, security, and records maintenance regulations and standards. Any cryptographic modules or data transmission modules will implement validated algorithms, ensure authentication, and maintain data integrity to ensure compliance with FIPS 140-3 Level 2, ISO/IEC 19790 Level 2, or equivalent data security standards. Although specific components are depicted, embodiments of the system 100 may include additional components or combine functionality among components depending on implementation requirements.

An AI conversational agent 110 is an artificial intelligence system configured to conduct structured and semi-structured clinical assessment interviews with a patient 102. In various embodiments, the AI conversational agent 110 is executed by software on the backend analysis servers 108, by an application on the patient's device 104, through an intermediary service such as Voiceflow or VAPI, or by a combination of software on the backend analysis servers 108 and an application on the patient's device 104 with or without integration with intermediary services. In some embodiments, the AI conversational agent 110 generates a speaking voice to engage the patient 102 in conversation using a service such as Elevenlabs, Deepgram, or an equivalent AI voice generation service.

The patient device 104 is a communications device operated or accessible by the patient 102 and configured to host or interface with the AI conversational agent 110. The patient device 104 may include, as embodiments, a mobile phone, telephone, tablet, laptop computer, desktop computer, wearable device, virtual home assistant, or other communications hardware capable of supporting audio communications. Any embodiment comprising a wearable device will utilize validated known low-risk design materials which minimize toxicity and produce negligible irritation to ensure compliance with ISO 10993, GB/T 16886, or equivalent device safety standards. The patient device 104 may execute a client application, software module, or other patient interface module that facilitates the communication session, manages local interactions with the AI conversational agent 110, and manages user-facing interface elements such as privacy notifications or feedback prompts. Any patient interface module will be configured for the intended user profile of applicable patients, will include design features that mitigate reasonably foreseeable misuse, and will remain safe despite exposure to common hazards to ensure compliance with IEC 62366, IEC 60601, or equivalent device usability standards. The patient device 104 may establish secure communication channels with cloud-based AI components and backend analysis servers 108, enabling encrypted transmission of session records 114.

Audio-capture components 106 are one or more sensors, microphones, or audio-processing components associated with the patient device 104 and configured to obtain speech data from the patient 102 during the assessment session. In some embodiments, the audio-capture components 106 may be integrated directly into the patient device 104, such as telephone microphones. In other embodiments, the audio-capture components 106 may include external or dedicated audio devices such as wearable microphones, wired or wireless headsets, or other standalone audio-capture modules that interface with the patient device 104 via wired connections, Bluetooth, or wireless communication protocols. The audio-capture components 106 may incorporate adaptive noise-isolation, echo-cancellation, or source-separation technology to improve audio capture quality and ensure that speech data remains suitable for downstream analysis. The audio-capture components 106 may further provide real-time audio quality indicators, detect environmental noise conditions, and apply noise-compensation filters.

Backend analysis servers 108 include one or more cloud-based or hardware-hosted processing devices. In some embodiments, the backend analysis servers 108 include a non-transitory computer-readable medium comprising instructions that cause one or more processing devices of the server(s) to implement the acts of the AI conversational agent, analyze audio data, generate time-aligned transcripts, extract speech-derived features, and generate cognitive assessment scores. The backend analysis servers 108 include an attribution module which generates transparent explanatory data detailing its cognitive assessment score generation (using SHAP or LIME, as exemplary methods) to ensure compliance with the EU AI Act, FDA PCCP, FDA Artificial Intelligence-Enabled Device Software Functions guidance, YY/T 1833, GB/T 45654, or equivalent Artificial Intelligence system design and security regulations and standards. The backend analysis servers 108 further comprise human-in-the-loop logging and algorithmic modification protocols to ensure compliance with the EU AI Act, FDA PCCP, FDA Artificial Intelligence-Enabled Device Software Functions guidance, YY/T 1833, GB/T 45654, or equivalent Artificial Intelligence system design and security regulations and standards. In some embodiments, the backend analysis servers 108 may enforce security controls and provide access via APIs or web-based portals with graphical user interfaces for transmitting assessment results to authorized recipients or clinician-accessible locations to ensure compliance with HL7 Fast Healthcare Interoperability Resources, HIPAA, YY/T 1843, GDPR, or equivalent medical data privacy and security regulations and standards.

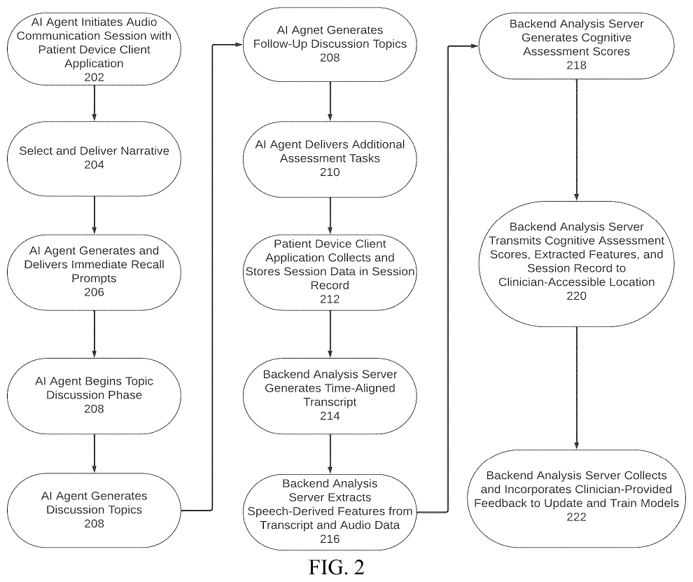

FIG. 2 is a flow diagram illustrating an example method 200 for conducting an AI-driven cognitive assessment communication session. Although specific steps are depicted, embodiments of the method 200 may include additional steps or combine functionality among steps depending on implementation requirements.
At step 202, an AI conversational agent initiates an audio communication session with the patient via the patient's device 202. In some embodiments, the communications session is configured to use a communications link coupled with a client application on the patient's device 202. In other embodiments, the communications session is configured to only use the native communications features present on the patient's device 202. The communication session may be scheduled, automatically triggered by predefined conditions, or patient-prompted.
At step 204, the AI conversational agent delivers a narrative selected from a library of relevant cognitive-assessment narratives. The selection may be based on patient metadata including demographics or determination of existing impairment from impairment indicators provided by the ordering physician. Delivery of the narrative utilizes audio components on the patient's device 202, including microphones and speakers.
At step 206, the agent queries the patient with an immediate recall free-response prompt to assess short-term memory retrieval. Delivery of the prompt utilizes audio components on the patient's device 202, including microphones and speakers.
At step 208, the AI conversational agent engages the patient in a topic discussion phase. Broad discussion topics are presented to the patient (e.g., “Tell me about your day”), followed by between one and ten follow-up questions based on the patient's response to the initial broad discussion topic. Multiple discussion topics are presented to the patient to elicit speech detailed enough for analysis. If the patient does not generate enough volume of speech, the agent will prompt the patient with further discussion topics until responses are sufficient for analysis, calculated based on the number of tokens generated by the patient per discussion topic (minimum 150 tokens, equivalent to approximately 100 words) as well as the duration of the total session (minimum 10 minutes). The discussion phase is designed to elicit spontaneous speech and conversational behavior for analysis, and follow-up queries may be generated in real time to maintain a natural conversational flow. This multi-stage relevance-driven topic generation architecture improves structural stability and increases the reliability of downstream scoring and assessment by improving speech collection under variable linguistic and situational conditions. Delivery of the topic discussion utilizes audio components on the patient's device 202, including microphones and speakers.
At step 210, the AI conversational agent delivers additional assessment tasks, including but not limited to delayed recall prompts to assess long-term memory recall or recognition or fluency tasks including categorical or letter verbal fluency. The patient's responses provide data indicative of cognitive functioning following a brief conversational distraction. Delivery of additional assessment tasks utilizes audio components on the patient's device 202, including microphones and speakers.
At step 212, session data is collected and stored in a session record on the patient's device 202. In some embodiments, a client application on the patient's device 202 controls the process of saving the session record. The collected session data may include audio from all conversational phases, patient responses, recall results, and metadata related to environmental conditions or device performance.
At step 214, a backend analysis server uses an Automatic Speech Recognition (ASR) model to process the audio data to produce a time-aligned transcript. In some embodiments, the ASR model is implemented by the backend analysis server using its processor, memory, and storage. In other embodiments, an external service provider implements the ASR model. Transcript words are associated with corresponding timestamps to facilitate alignment with acoustic features and conversational segments.
At step 216, a backend analysis server uses a feature-embedding model to extract a plurality of speech-derived features from the audio data and corresponding time-aligned transcript, including prosodic, acoustic, lexical, and semantic-coherence metrics. In some embodiments, the feature-embedding model is implemented by the backend analysis server using its processor, memory, and storage. In other embodiments, an external service provider implements the feature-embedding model. In some embodiments, the model comprises multiple feature-processing stages to separately extract audio characteristics and transcript-derived linguistic features. Extracted features may include articulation rates, speech segmentation, lexical frequencies and diversities, parts-of-speech usage and ratios, disfluency and pause rates and durations, and other measures relevant to cognitive analysis. In certain embodiments, feature extraction models may include methods such as tagging parts-of-speech, linguistic analysis, audiometric analysis, tagging function and content words, identifying speech error/repair markers, applying LSTM, BERT, or other models to generate embeddings from recorded and transcribed speech, or equivalent feature extraction methods. Semantic-coherence metrics may include cosine similarities between consecutive utterance embeddings, a cumulative topic-drift measure of the difference between early-session and late-session embedding features, graph-based coherence measures derived from an utterance similarity graph, or equivalent semantic-coherence metrics. Analysis of semantic-coherence metrics will also generate content and memory features, including but not limited to forgetting rates for content units (names, locations, and other details), accuracy and breadth of descriptions, and integration of acoustic and lexical features (e.g., duration of unfilled pauses in descriptions of location content units).
At step 218, a backend analysis server uses a machine-learning classifier trained on clinically validated datasets to generate one or more cognitive assessment scores by comparing extracted features to diagnostic indicators. In some embodiments, the machine-learning classifier comprises a multi-stream, multi-stage architecture. Input streams may include data separated by modality (e.g., lexical data derived from transcripts vs audiometric data derived from waveforms, etc.), by task (e.g., narrative recall data indicating memory performance, interview data indicating presence or absence of cognitive leisure activities, etc.), or a combination of the two. The first stage of analysis integrates each input stream into specialized subsets of models based on domains and subdomains (e.g., memory, attention, language use, social functioning, physical activity, cognitive activity, etc.), with features assigned to each model subset based on a combination of domain expertise, feature selection, feature engineering, and/or dimensionality reduction. These first-stage models will use multi-model multi-modal pipelines that integrate architectures including but not limited to random forests with and without gradient boosting, support vector machines, logistic and linear regressions, and neural networks. Each input stream may also be processed by multiple stages of models to produce domain and subdomain subscores. A second stage will integrate the outputs of these models (final layer for multilayer perceptrons, confidence scores for random forest or logistic regression classifiers, regression scores for regression models, etc.) in combination with other engineered features to further refine the collected data into explainable subscores to support impairment classification and risk assessment. In some embodiments, the generated assessment scores following this stage may include impairment probabilities, risk scores, cognitive domain scores, deviation indices, or other clinically relevant metrics, each based on outputs derived from first stage subscores, second stage subscores, or a combination of the two. In some embodiments, the backend analysis server may fuse the multiple input streams using a feature-fusion model that applies dynamic weights to each feature type based on its learned relevance to impairment assessment prediction. The backend analysis server may apply several regression or classification models, including but not limited to logistic or linear regressions, support vector machines, random forests, gradient-boosted machines, and neural networks including multilayer perceptrons or transformer models. The multi-stream and fusion-based architecture improves robustness and enhances consistency of impairment assessments across conversational situations relative to single-stream or non-fused classifications.
At step 220, the cognitive assessment scores, extracted features, and session records are transmitted to a clinician-accessible location, which may include a web portal with a graphical user interface, an electronic health record (EHR) system, a clinical analytics platform, a caregiver device, or a backend storage system. Data transmissions are facilitated using the backend analysis server's network components, which may include, but are not limited to, Network Interface Cards, optical transceivers, wireless communications transceivers, VSAT terminals, and other equivalent network components. In some embodiments, a clinician may access transmitted scores, features, and records using a computing device such as a tablet, mobile phone, computer, or other clinician-accessible device. In some embodiments, transmission of cognitive assessment scores may include an alert or other indicator provided to the clinician-accessible location to notify the clinician that the patient's scores warrant follow-up clinician assessment.
At step 222, some embodiments of the system may collect and incorporate clinician-provided cognitive assessment scores and diagnoses of cognitive or neurodegenerative disorders into downstream model-update procedures, perform population-level assessment comparisons, or initiate follow-up assessments.

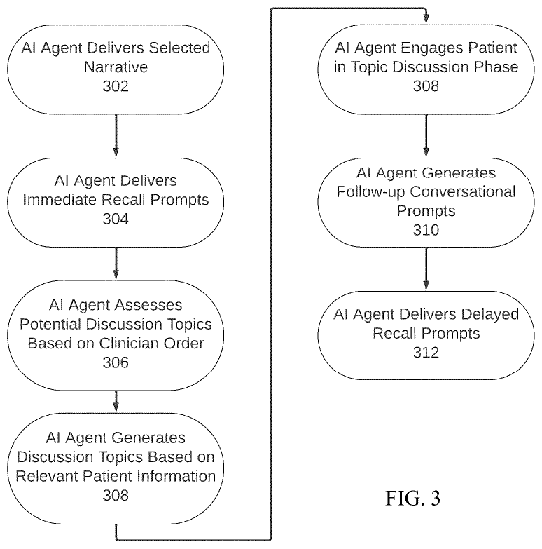

FIG. 3 is a flow diagram illustrating an example process 300 for generating and delivering discussion topics based on patient-specific contextual data. Although specific steps are depicted, embodiments of the process 300 may include additional steps or combine functionality among steps depending on implementation requirements.
At step 302, an AI conversational agent delivers a narrative selected from a library of clinically relevant cognitive-assessment narratives to the patient. Narrative selection may be based on patient metadata including demographics or determination of existing impairment from indicators provided by the ordering physician.
At step 304, an AI conversational agent queries the patient's retention of immediate recall prompt information is queried to evaluate short-term memory function using an open-ended free recall question (e.g., “Describe everything you remember about the story”), which is statically linked to the delivered narrative to maintain consistency across patient interactions. The AI conversational agent queries the patient's information retention using the audio-capture components on the patient's device.
At step 306, an AI conversational agent identifies potential discussion topics that align with assessment objectives based on the type of evaluation ordered by the clinician.
At step 308, an AI conversational agent generates one or more discussion topics for the topic-discussion phase. These topics may concern the patient's hobbies, family matters, occupation, daily activities, well-being, diet, physical activity, social activity, personal opinions, or other previously identified conversational themes. The AI conversational agent delivers discussion topics using the audio-capture components on the patient's device.
At step 310, an AI conversational agent dynamically generates follow-up conversational prompts, enabling the AI conversational agent to maintain a natural conversation while eliciting speech samples relevant to cognitive assessment. The AI conversational agent delivers follow-up prompts using the audio-capture components on the patient's device.
At step 312, following the topic-discussion phase, the AI conversational agent queries the patient's retention of delayed recall prompt information to evaluate long-term memory function using open-ended free recall (“Describe everything you remember about the story”), as well as targeted prompts about specific narrative units (“Do you remember the name of the dog?”). The recall prompts are statically linked to the delivered narrative to maintain consistency across patient interactions. The AI conversational agent queries the patient's information retention using the audio-capture components on the patient's device.

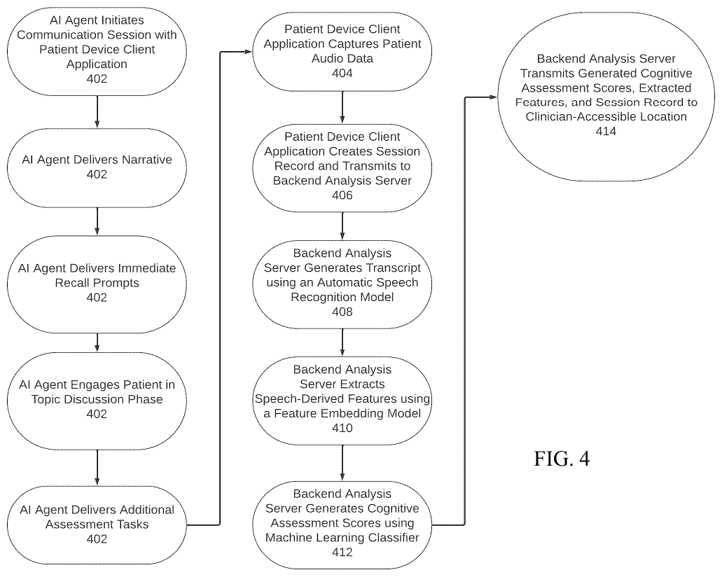

FIG. 4 is a flow diagram of an example method 400 for generating cognitive assessment scores from patient speech. Although specific method steps are depicted, embodiments of the method 400 may include additional steps or combine functionality among steps depending on implementation requirements.
At step 402, an audio communication session between the AI conversational agent and the patient is initiated. In some embodiments, the communications session is configured to use a communications link coupled with a client application on the patient's device. In other embodiments, the communications session is configured to only use the native communications features on a patient-accessible device. During the session, the AI conversational agent delivers a selected narrative, presents recall prompts, and engages in a topic-discussion phase to collect speech data.
At step 404, audio-capture components associated with the patient device captures patient audio. The audio-capture components may apply one or more adaptive noise-compensation or signal-enhancement techniques as needed to prepare the audio for analysis, including a Wiener filter, spectral subtraction filter, or a deep learning-based noise suppression model.
At step 406, session data is collected and stored in a session record and transmitted to backend servers for analysis using communications components on the patient's device. In some embodiments, a client application on the patient's device manages saving and transmitting the session record. The session record may include patient responses, task results, and metadata related to environmental conditions or device performance.
At step 408, the backend analysis servers use an Automatic Speech Recognition (ASR) model to process the session record and generate a time-aligned transcript containing words or tokens associated with corresponding audio timestamps. In some embodiments, the ASR model is implemented by the backend analysis server using its processor, memory, and storage. In other embodiments, an external service provider implements the ASR model.
At step 410, the backend analysis servers employ a feature-embedding model to extract a plurality of speech-derived features from the audio data and transcript. These speech-derived features may include prosodic, audiometric, lexical, and semantic-coherence features. In some embodiments, the feature-embedding model is implemented by the backend analysis server using its processor, memory, and storage. In other embodiments, an external service provider implements the feature-embedding model.
At step 412, the backend analysis servers use a machine-learning classifier trained using clinically validated and labeled patient data from one or more datasets to analyze extracted features and generate one or more cognitive assessment scores indicative of neurological, psychiatric, or cognitive conditions, including risk probabilities or impairment metrics.
At step 414, the backend analysis servers transmit the generated scores, extracted features, and session record to one or more clinician-accessible location. These clinician-accessible location may include clinical electronic health record (EHR) platforms, secure storage repositories, a web portal with a graphical user interface, or clinician-facing review tools. Data transmissions are facilitated using the backend analysis server's network components, which may include, but are not limited to, Network Interface Cards, optical transceivers, wireless communications transceivers, VSAT terminals, and other equivalent network components. In some embodiments, a clinician may access transmitted scores, features, and records using a computing device such as a tablet, mobile phone, computer, or other clinician-accessible device. In some embodiments, transmission of cognitive assessment scores may include an alert or other indicator provided to the clinician-accessible location to notify the clinician that the patient's scores warrant follow-up clinician assessment. In some embodiments, clinician feedback may be incorporated into iterative model-update workflows.

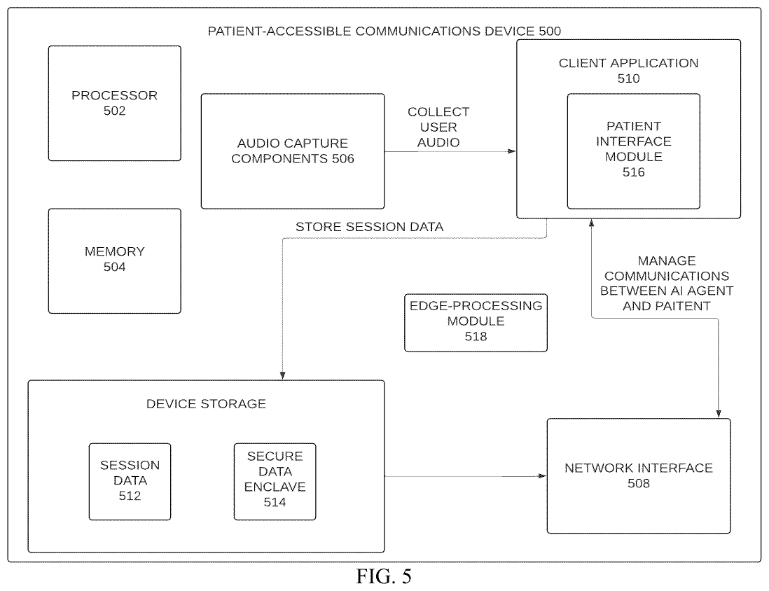

FIG. 5 is a block diagram of an example patient-accessible communications device 500 configured to execute or interface with the AI conversational agent. The device 500 may include a processor 502, memory 504, audio-capture components 506, and a network interface 508 configured to communicate with an AI-based assessment system via one or more networks. In some embodiments, the device 500 executes a client application 510 to facilitate the cognitive assessment session, manage communications with the AI conversational agent, manage device data operations, and provide user-facing controls and notifications. Although specific device features are depicted, embodiments of the device 500 may include additional features or combine functionality among features depending on implementation requirements.

The audio capture components 506 of the device 500 may include one or more microphones, audio sensor arrays, or external audio-capture peripherals that interface with the device via wired connectors, Bluetooth, or other wireless protocols. The audio-capture components 506 may incorporate noise-isolation, echo-cancellation, or other audio quality improvement capabilities to ensure that patient speech is captured with sufficient fidelity for downstream analysis.

In some embodiments, the client application 510 on the device 500 may store session data 512, including buffered audio samples and local metadata used to maintain session continuity. The device 500 includes a secure data enclave 514 used for encryption, authentication, or privacy-secure processing of patient data. All patient data will be encrypted and maintained in an access-controlled, audit-enabled secure directory to ensure compliance with HIPAA, FDA ERES, SP 800-111, SP 800-122, YY/T 1843, GDPR, or equivalent healthcare data privacy, security, and records maintenance regulations and standards. Any cryptographic modules or data transmission modules will implement validated algorithms, implement authentication controls, and maintain data integrity to ensure compliance with FIPS 140-3 Level 2, ISO/IEC 19790 Level 2, or equivalent data security standards. All session data 512 and data within the secure data enclave 514 are removed from the device 500 at the conclusion of each session using cryptographic erasure methods compliant with SP 800-88 or equivalent media sanitization standards. In some embodiments, a patient interface module 516 within the client application 510 may present instructions, progress indicators, privacy statements, or optional feedback prompts to the patient. The device 500 may further support integration with wearable devices or smart-home devices configured to augment or enhance audio capture during the assessment. The device 500 may further include a localized edge-processing module 518 to perform region-specific patient data processing to comply with resident processing requirements.

What is claimed is:

1. A method for generating cognitive assessment scores from patient speech, the method comprising:
initiating, by a server hosting an artificial intelligence (“AI”) conversational agent, an audio communication session with a patient;
delivering to the patient, by the AI conversational agent, at least one oral narrative selected from a library of clinically validated narratives;
delivering to the patient, by the AI conversational agent, one or more oral cognitive assessments, including but not limited to categorical or letter verbal fluency, short-term memory word recall, text descriptions, and picture descriptions;
delivering to the patient, by the AI conversational agent, one or more immediate recall audio prompts for the patient to orally recall information from the oral narrative;
engaging the patient, by the AI conversational agent, in an oral topic discussion phase incorporating targeted follow-up questions dynamically generated based on the patient's responses;
after engaging in an oral topic discussion phase, delivering to the patient, by the AI conversational agent, one or more delayed recall audio prompts for the patient to orally recall information from the oral narrative;
collecting a collection of audio data retrieved during the audio communication session;
generating, using an automatic speech recognition (ASR) model, a time-aligned transcript from the collection of audio data, wherein words in the transcript are associated with corresponding audio data timestamps of the collection of audio data;
extracting, using a feature-embedding model, a plurality of speech-derived features from the collection of audio data and time-aligned transcript;
wherein the plurality of speech-derived features comprise but are not limited to prosodic features, acoustic characteristics, lexical features, and semantic-coherence metrics;
generating at least one cognitive assessment score from the plurality of speech-derived features;
wherein generating the at least one cognitive assessment score includes comparing the plurality of speech-derived features to diagnostic indicators;
wherein the diagnostic indicators are generated by a machine-learning classifier trained on at least one clinically validated dataset; and
transmitting the at least one cognitive assessment score to a clinician-accessible device.

2. The method of claim 1, wherein selection of the at least one oral narrative is based on patient metadata including demographics or existing impairment indicators provided by an ordering physician.

3. The method of claim 1, wherein the one or more immediate and delayed audio recall prompts relate to semantic units comprising settings, characters, events, and thematic elements extracted from the oral narrative.

4. The method of claim 1, wherein the oral topic discussion phase conducted by the AI conversational agent includes oral discussion topics generated based on patient responses and metadata, and in which follow-up queries are generated based on previous patient responses, and wherein oral discussion topics are generated from the group consisting essentially of the patient's hobbies, family matters, occupation, daily activities, wellbeing, and personal opinions.

5. The method of claim 1, wherein a backend analysis server obtains patient session feedback prior to termination of the audio communication session.

6. The method of claim 1, wherein a session record is generated from audio communication session data comprising collected audio data from the one or more cognitive assessment, device diagnostic information, patient identifying information, session metadata, environmental information, cognitive assessment results, topic-phase data, device information, environmental data, session metadata, and patient identifiers.

7. The method of claim 1, wherein a feature-embedding model is configured to generate feature vectors comprising articulation rates, speech segmentation, voice onset time, Mel-frequency Cepstral Coefficients, fundamental frequency, jitter, shimmer, contextual relevance metrics, harmonics-to-noise ratio, and formant.

8. The method of claim 1, wherein a machine-learning classifier is trained to generate at least one cognitive assessment score for the measurement and prediction of disorders and conditions including but not limited to Mild Cognitive Impairment, Alzheimer's Disease, preclinical Alzheimer's Disease, age-related cognitive decline, Vascular Dementia, Dementia with Lewy Bodies, Frontotemporal Dementia, Parkinson's Disease with or without Dementia, Depression, Stroke, Huntington's Disease, Fatal Familial Insomnia, Traumatic Encephalopathy, and Normal Pressure Hydrocephalus.

9. The method of claim 1, wherein the generated at least one cognitive assessment score includes at least one of an impairment probability score, risk of impairment probability score, or an impairment etiology (e.g., Alzheimer's disease, vascular contributions, depression) probability score.

10. The method of claim 1, wherein the generated at least one cognitive assessment score includes at least one of a lexical complexity score, fluency control score, lexical diversity score, grammatical sophistication score, executive control score, descriptiveness score, content accuracy score, coherence score, and scores for cognitive domains including memory, executive functioning, language use, visuospatial processing, and attention.

11. The method of claim 1, wherein the generated at least one cognitive assessment score includes transparent explanatory data and human-in-the-loop control elements compliant with the EU AI Act, FDA PCCP, YY/T 1833, or equivalent Artificial Intelligence system design and security regulations and standards.

12. The method of claim 1, wherein transmitted data includes standardized clinical data fields compliant for integration with an electronic health records system.

13. The method of claim 1, wherein data erasure complies with SP 800-88 or equivalent media sanitization standards.

14. The method of claim 1, wherein integrations with electronic health records systems comply with HL7 Fast Healthcare Interoperability Resources standards, HIPAA, YY/T 1843, GDPR, or equivalent medical data privacy and security regulations and standards.

15. The method of claim 1, wherein collected and transmitted data is encrypted using cryptographic modules compliant with FIPS 140-3 Level 2 or equivalent data security standards.

16. The method of claim 1, wherein a feedback-based training procedure is executed using clinician-generated feedback and diagnosis data to update parameters of at least one model used by the method.

17. A non-transitory computer-readable medium comprising:
a non-transitory computer readable medium including instructions which, when executed by one or more processing device, initiating the one or more processing device to:
implement an artificial intelligence (“AI”) based cognitive assessment system that is configured to:
initiate, using a server hosting an AI conversational agent, an audio communication session with a patient;
deliver to the patient, using the AI conversational agent, an oral narrative selected from a library of clinically validated narratives;
deliver to the patient, using the AI conversational agent, one or more oral cognitive assessments, including but not limited to categorical or letter verbal fluency, short-term memory word recall, text descriptions, and picture descriptions;
deliver to the patient, using the AI conversational agent, one or more immediate recall audio prompts for the patient to orally recall information from the oral narrative;
engage the patient, using the AI conversational agent, in an oral topic discussion phase;
wherein oral discussion topics for the topic-discussion phase are generated by the AI conversational agent based on patient responses and metadata;
wherein targeted follow-up queries are dynamically generated by the AI conversational agent based on the patient's responses;
deliver to the patient, using the AI conversational agent, one or more delayed recall audio prompts for the patient to orally recall information from the oral narrative;
collect a collection of audio data retrieved during the audio communication session;
generate, using an automatic speech recognition (ASR) model, a time-aligned transcript from the collection of audio data;
wherein transcript words are associated with corresponding audio data timestamps of the collection of audio data;
extract, using a feature-embedding model, a plurality of speech-derived features from the collection of audio data and time-aligned transcript;
wherein the plurality of speech-derived features comprise, but are not limited to, prosodic features, acoustic characteristics, lexical features, and semantic-coherence metrics;
generate, using a machine-learning classifier, at least one cognitive assessment score from the plurality of speech-derived features;
wherein assessment score generation includes comparing the plurality of speech-derived features to diagnostic indicators;
wherein the machine-learning classifier is trained on clinically validated datasets; and
transmit, using a communication interface, the cognitive assessment score to a clinician-accessible device.

18. The non-transitory computer-readable medium of claim 17, wherein selection of the oral narrative is based on patient metadata including demographics or existing impairment indicators provided by an ordering physician.

19. The non-transitory computer-readable medium of claim 17, wherein oral topic discussions conducted by the AI conversational agent include oral discussion topics generated based on patient responses and metadata, and in which follow-up queries are generated based on previous patient responses, and wherein oral discussion topics are generated from the group consisting essentially of the patient's hobbies, family matters, occupation, daily activities, wellbeing, and personal opinions.

20. The non-transitory computer-readable medium of claim 17, wherein a feature-embedding model is configured to generate feature vectors comprising articulation rates, speech segmentation, voice onset time, Mel-frequency Cepstral Coefficients, fundamental frequency, jitter, shimmer, contextual relevance metrics, harmonics-to-noise ratio, and formant.

21. The non-transitory computer-readable medium of claim 17, wherein a machine-learning classifier is trained to generate at least one cognitive assessment score for the measurement and prediction of disorders and conditions including but not limited to Mild Cognitive Impairment, Alzheimer's Disease, preclinical Alzheimer's Disease, age-related cognitive decline, Vascular Dementia, Dementia with Lewy Bodies, Frontotemporal Dementia, Parkinson's Disease with or without Dementia, Depression, Stroke, Huntington's Disease, Fatal Familial Insomnia, Traumatic Encephalopathy, and Normal Pressure Hydrocephalus.

22. The non-transitory computer-readable medium of claim 17, wherein the generated at least one cognitive assessment score includes at least one of an impairment probability score, risk of impairment probability score, or an impairment etiology (Alzheimer's disease, vascular contributions, depression) probability score.

23. The non-transitory computer-readable medium of claim 17, wherein the generated at least one cognitive assessment score includes at least one of a lexical complexity score, fluency control score, lexical diversity score, grammatical sophistication score, executive control score, descriptiveness score, content accuracy score, coherence score, and scores for cognitive domains including memory, executive functioning, language use, visuospatial processing, and attention.

24. The non-transitory computer-readable medium of claim 17, wherein the generated at least one cognitive assessment score includes transparent explanatory data and human-in-the-loop control elements compliant with the EU AI Act, FDA PCCP, YY/T 1833, or equivalent Artificial Intelligence system design and security regulations and standards.

25. The non-transitory computer-readable medium of claim 17, wherein transmitted data includes standardized clinical data fields compliant for integration with an electronic health records system.

26. The non-transitory computer-readable medium of claim 17, wherein data erasure complies with SP 800-88 or equivalent media sanitization standards.

27. The non-transitory computer-readable medium of claim 17, wherein integrations with electronic health records systems comply with HL7 Fast Healthcare Interoperability Resources standards, HIPAA, YY/T 1843, GDPR, or equivalent medical data privacy and security regulations and standards.

28. The non-transitory computer-readable medium of claim 17, wherein collected and transmitted data is encrypted using cryptographic modules compliant with FIPS 140-3 Level 2 or equivalent data security standards.

29. The non-transitory computer-readable medium of claim 17, wherein a session record is generated from audio communication session data comprising collected audio data from the one or more cognitive assessment, device diagnostic information, patient identifying information, session metadata, environmental information, cognitive assessment results, topic-phase data, device information, environmental data, session metadata, and patient identifiers.
